(19) United States
(12) Patent Application Publication
(10) Pub. No.: US 2026/0345067 A1
(43) Pub. Date: Nov. 5, 2026

(54) SMART EYEWEAR SYSTEM FOR REAL-TIME POSTURE CORRECTION FOCUS MONITORING AND COGNITIVE STRAIN MANAGEMENT

(71) Applicant: Ultrahuman Healthcare Private Limited, Bangalore South (IN)

(72) Inventors: Jyoti Lavania, Bangalore South (IN); Aditi Bhattacharya, Bangalore South (IN); Vatsal Singhal, Bangalore South (IN); Mohit Kumar, Bangalore South (IN)

(73) Assignee: Ultrahuman Healthcare Private Limited, Bangalore South (IN)

(21) Appl. No.: 19/714,196

(22) Filed: Jun. 19, 2026

(30) Foreign Application Priority Data
Jun. 19, 2025 (IN) 202541058876

(51) Int. Cl. A61B 5/00; A61B 5/11; A61B 5/16

(52) U.S. Cl. CPC A61B 5/4561; A61B 5/1116; A61B 5/163; A61B 5/6814; A61B 5/746

(57) ABSTRACT

A smart eyewear system for ergonomic cognitive strain management is disclosed. The smart eyewear system comprises a plurality of sensors integrated into an eyewear frame and configured to collect sensor data associated with posture conditions, neck positioning conditions, attention features, and interaction conditions associated with a user. A controller configured to processes the sensor data to generate synchronized posture features and attention features. The controller further checks availability of previously stored user specific baseline information, retrieves a previously stored user specific baseline when available, or determines a user specific baseline when unavailable. The controller determines an ergonomic cognitive strain state based on the synchronized posture features, the attention features, and the user specific baseline, and generates feedback cue associated with the ergonomic cognitive strain state for the smart eyewear system.

### FIELD OF INVENTION

The present disclosure generally relates to a wearable device. More specifically, the present disclosure is related to a smart wearable device capable of detecting and responding to ergonomic strain and attention loss.

### BACKGROUND OF THE INVENTION

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

The increasing adoption of digital work environments, remote working systems, and prolonged computer-based activities has significantly increased the amount of time users spend performing desk based tasks using laptops, desktop computers, tablets, and other electronic devices. Extended engagement in such activities often causes users to maintain improper ergonomic posture for prolonged durations, including forward head tilt, neck bending, shoulder imbalance, and slouched sitting positions. Continuous exposure to such unhealthy posture conditions may contribute to musculoskeletal strain, spinal discomfort, reduced productivity, fatigue, headaches, and long-term ergonomic disorders.

To address such concerns, several posture monitoring systems and wearable posture correction devices have been developed. Certain existing systems utilize inertial sensing devices or body mounted sensors configured to detect spinal orientation or body alignment deviations and generate corrective alerts when posture abnormalities are detected. Commercially available posture correction devices further provide vibration based notifications to encourage users to maintain upright posture during daily activities. However, such systems are primarily limited to posture tracking applications and generally do not evaluate user attention, cognitive engagement, gaze behavior, or mental fatigue during prolonged work sessions.

In parallel, multiple wearable wellness monitoring devices have been introduced for tracking general physiological and activity related parameters including sleep quality, heart rate, physical activity, respiratory behavior, metabolic performance, and recovery indicators. Such devices provide users with wellness summaries and physiological analytics associated with overall health conditions. However, existing wellness wearables generally do not address desk based ergonomic behavior, attention consistency, gaze disengagement, or cognitive strain associated with prolonged screen exposure and continuous digital work environments. Further, certain systems attempt to estimate focus levels or cognitive fatigue using physiological measurements, breath analysis, electroencephalography (EEG) based monitoring techniques, or task based cognitive assessments. Academic and research-oriented systems additionally evaluate mental fatigue through periodic testing procedures or specialized monitoring equipment. However, such approaches are often unsuitable for continuous real-time daily usage due to hardware complexity, user inconvenience, lack of wearable integration, or inability to provide immediate corrective intervention during occurrence of cognitive degradation events.

Existing technologies further suffer from fragmented operation because posture monitoring, wellness tracking, focus estimation, and fatigue assessment are generally implemented as independent systems operating without coordinated analysis. Consequently, existing solutions are unable to establish relationships between ergonomic posture, gaze engagement, cognitive focus, and user fatigue conditions during real world work activities. Additionally, many conventional systems rely upon static threshold based evaluation techniques that fail to adapt according to individual user behavior, environmental conditions, work patterns, or personalized ergonomic baselines.

Several existing systems additionally operate primarily as passive tracking platforms that generate retrospective reports after completion of user activities. Such systems often fail to provide proactive and context aware feedback capable of assisting users during occurrence of unhealthy ergonomic or cognitive strain conditions. As a result, corrective actions are delayed and users may continue operating under suboptimal physical and mental conditions for extended durations.

Accordingly, there exists a need for an integrated wearable monitoring system capable of continuously evaluating ergonomic posture, gaze behavior, cognitive engagement, and mental fatigue using multiple sensing modalities in real-time. Further, there exists a need for a personalized intelligent monitoring system capable of adaptively learning user specific behavioral patterns and proactively generating corrective feedback to improve ergonomic wellness, attention consistency, cognitive performance, and long-term behavioral habits during prolonged desk based activities.

### OBJECTS OF THE INVENTION

An object of the present disclosure is to provide a wearable monitoring system configured to continuously monitor ergonomic posture during desk-based activities.

Another object of the present disclosure is to provide smart eyewear configured to evaluate neck positioning, head orientation, and posture related strain using inertial and pressure-based sensing mechanisms.

Another object of the present disclosure is to provide a smart eyewear system capable of evaluating cognitive strain associated with prolonged screen exposure, blue light exposure, gaze behavior, attention drift, and user engagement patterns.

Another object of the present disclosure is to provide a gaze or attention monitoring module configured to continuously generate attention features associated with user focus and screen engagement.

Another object of the present disclosure is to provide a controller configured to generate synchronized posture and attention features using data obtained from multiple sensing modalities.

Another object of the present disclosure is to provide a sensor fusion framework configured to combine pressure features, inertial features, gaze features, blue light exposure parameters, and attention features to determine an ergonomic cognitive strain state associated with a user.

Another object of the present disclosure is to provide a wearable system configured to generate real-time eyewear integrated feedback cues in response to detection of ergonomic strain, cognitive fatigue, posture deviation, or attention degradation conditions.

Another object of the present disclosure is to provide a personalized monitoring system configured to adapt a user specific baseline over time according to historical behavioral patterns, ergonomic tendencies, attention characteristics, and environmental conditions.

Another object of the present disclosure is to provide a real-time intervention system capable of proactively assisting users before prolonged ergonomic or cognitive degradation conditions occur.

### SUMMARY OF THE INVENTION

This summary is provided to introduce aspects related to a smart eyewear system for ergonomic cognitive strain management and the aspects are further described below in the detailed description. This summary is not intended to identify essential features of the claimed subject matter nor is it intended for use in determining or limiting the scope of the claimed subject matter.

In an embodiment, the present disclosure provides a smart eyewear system configured to monitor posture conditions, neck positioning conditions, attention features, and interaction conditions associated with a user. The smart eyewear system comprises a plurality of sensors integrated into an eyewear frame and configured to collect sensor data associated with posture conditions, neck positioning conditions, attention features, and interaction conditions associated with the user. A controller is connected with the plurality of sensors and configured to generate synchronized posture features and attention features, check availability of previously stored user specific baseline information corresponding to the user, retrieve a previously stored user specific baseline when the previously stored user specific baseline information is available, or determine a user specific baseline when the previously stored user specific baseline information is unavailable, determine an ergonomic cognitive strain state using the synchronized posture features, the attention features, and the user specific baseline, and generate feedback cue associated with the ergonomic cognitive strain state and the user specific baseline for output at the smart eyewear system.

In one aspect, the smart eyewear system further comprises a wireless module configured to communicate the feedback cue to a user device.

In one aspect, the wireless module is configured to communicate at least one of sensor data, processed sensor data, or monitoring information to the user device.

In one aspect, the user device communicates the at least one of the sensor data, processed sensor data, and monitoring information to a remote server periodically.

In one aspect, the smart eyewear system further comprises a memory configured to store sensor data, processed sensor data, historical posture conditions, attention features, ergonomic cognitive strain states, behavioral patterns, user specific baseline information, or monitoring information associated with the user.

In one aspect, the controller is configured to retrieve the user specific baseline information from the memory.

In one aspect, the feedback comprises at least one of a visual indication, an audio indication, a haptic indication, a notification, an alert, a posture correction recommendation, a fatigue indication, an attention reminder, a blue light exposure warning, or a user guidance output associated with the ergonomic cognitive strain state.

In one aspect, the controller is configured to update the user specific baseline using historical posture conditions, attention conditions, ergonomic cognitive strain states, behavioral patterns, and user interaction conditions associated with the user.

In one aspect, the plurality of sensors comprises at least one inertial sensing sensor configured to generate inertial data associated with head orientation, neck positioning, head tilt, and posture related movement conditions associated with the user, and a plurality of pressure sensors positioned at eyewear user contact regions including a nose bridge region and ear contact portions associated with the eyewear frame.

In one aspect, the plurality of pressure sensors is configured to generate pressure data associated with posture induced loading conditions, pressure distribution characteristics, and ergonomic strain conditions associated with the user.

In one aspect, the plurality of sensors further comprises at least one gaze or attention monitoring sensor configured to generate gaze related data and attention related data associated with gaze behavior, screen engagement patterns, and attention characteristics associated with the user, and at least one blue light exposure monitoring sensor configured to generate blue light exposure related data associated with prolonged screen interaction activities associated with the user.

In one aspect, the plurality of sensors further comprises one or more pressure sensors positioned at a nose bridge region and ear contact portions associated with the eyewear frame to generate pressure data, at least one inertial sensing sensor configured to generate inertial data associated with head orientation and neck positioning conditions associated with the user, and at least one gaze or attention monitoring sensor configured to generate gaze related data and attention related data associated with the user, wherein the controller is configured to temporally align and fuse the pressure data, the inertial data, the gaze related data, and the attention related data to determine the ergonomic cognitive strain state associated with the user and generate the feedback cue based on the ergonomic cognitive strain state and the user specific baseline.

In one aspect, the controller is configured to temporally align sensor data to generate synchronized posture features and attention features and fuse the synchronized posture features and attention features to determine the ergonomic cognitive strain state associated with the user.

In one aspect, the ergonomic cognitive strain state comprises at least one of a posture related strain condition, a neck positioning condition, a cognitive strain condition, an attention degradation condition, or a blue light exposure condition associated with the user.

In one aspect, the controller is configured to adapt the user specific baseline by dynamically adjusting one or more threshold values associated with posture characteristics, attention characteristics, or ergonomic cognitive strain characteristics based on historical sensor data and user interaction conditions associated with the user.

In one aspect, the feedback comprises at least one of a haptic feedback cue, a visual feedback cue, and an audio feedback cue configured to provide a real-time corrective notification associated with the ergonomic cognitive strain state.

In one aspect, the monitoring information communicated to the user device comprises posture related information, ergonomic cognitive strain information, attention related information, blue light exposure related information, and historical behavioral information associated with the user.

In one aspect, the controller is configured to perform on device processing of the sensor data associated with the user.

In one aspect, the plurality of sensors further comprises at least one ambient light sensing sensor configured to generate environmental lighting data associated with a surrounding environment of the user.

In one aspect, the plurality of sensors further comprises at least one electroencephalography sensing sensor configured to generate cognitive activity related data associated with the user.

In one aspect, the smart eyewear system is configured to continuously monitor ergonomic posture conditions and cognitive strain conditions associated with prolonged desk based activities performed by the user.

In one aspect, the pressure data comprises pressure distribution data and pressure differential data associated with the one or more pressure sensors, wherein the controller is configured to determine posture imbalance conditions and neck positioning conditions using the pressure distribution data and the pressure differential data, wherein greater pressure at a left nose contact region relative to a right nose contact region or greater pressure at the right nose contact region relative to the left nose contact region indicates head tilt conditions or lateral imbalance conditions, and increased pressure at the nose bridge region combined with reduced pressure at ear contact portions indicates forward head posture conditions, neck inclination conditions, or slouching conditions associated with the user.

In one aspect, the controller is configured to establish one or more threshold values during a calibration period using sensor data corresponding to normal user behavior or preferred ergonomic conditions collected over a predetermined duration.

In one aspect, the controller is configured to dynamically adjust the one or more threshold values according to confidence scores associated with reliability characteristics of the sensor data, wherein sensor data having low confidence scores, inconsistent measurements, or transient anomalies are excluded or assigned reduced weighting during threshold adaptation operations.

In an aspect, the present disclosure further provides a method for ergonomic cognitive strain management using the smart eyewear system. The method comprises collecting sensor data associated with posture conditions, neck positioning conditions, attention features, and interaction conditions associated with the user, processing the sensor data to generate synchronized posture features and attention features, checking whether previously stored user specific baseline information corresponding to the user is available, retrieving a previously stored user specific baseline when the previously stored user specific baseline information is available, or determining a user specific baseline when the previously stored user specific baseline information is unavailable, determining an ergonomic cognitive strain state using the synchronized posture features, the attention features, and the user specific baseline, and generating a feedback cue based on the ergonomic cognitive strain state and the user specific baseline, and outputting the feedback cue at the smart eyewear system.

In one aspect, the method further comprises communicating, through a wireless module, the feedback cue to a user device.

In one aspect, the method further comprises generating pressure data using one or more pressure sensors positioned at a nose bridge region and ear contact portions associated with an eyewear frame, generating inertial data using at least one inertial sensing sensor, generating gaze related data and attention related data using at least one gaze or attention monitoring sensor, temporally aligning and fusing the pressure data, the inertial data, the gaze related data, and the attention related data to determine the ergonomic cognitive strain state associated with the user, and generating the feedback cue based on the ergonomic cognitive strain state and the user specific baseline.

In one aspect, the method further comprises generating pressure distribution data and pressure differential data associated with the pressure data and determining posture imbalance conditions and neck positioning conditions using the pressure distribution data and the pressure differential data, wherein greater pressure at a left nose contact region relative to a right nose contact region or greater pressure at the right nose contact region relative to the left nose contact region indicates head tilt conditions or lateral imbalance conditions, and increased pressure at the nose bridge region combined with reduced pressure at ear contact portions indicates forward head posture conditions, neck inclination conditions, or slouching conditions associated with the user.

In one aspect, the method further comprises adapting the user specific baseline by dynamically adjusting one or more threshold values associated with posture characteristics, attention characteristics, or ergonomic cognitive strain characteristics based on historical sensor data and user interaction conditions associated with the user.

In one aspect, the method further comprises establishing the one or more threshold values during a calibration period using sensor data corresponding to normal user behavior or preferred ergonomic conditions collected over a predetermined duration.

In one aspect, the method further comprises adjusting the one or more threshold values according to confidence scores associated with reliability characteristics of the sensor data, wherein sensor data having low confidence scores, inconsistent measurements, or transient anomalies are excluded or assigned reduced weighting during threshold adaptation operations.

In one aspect, the plurality of sensors further comprises at least one electroencephalography sensing sensor configured to generate cognitive activity related data associated with the user, wherein the cognitive activity related data corresponds to neural activity patterns, attention related activity, cognitive workload characteristics, fatigue related activity, stress related activity, concentration characteristics, or combinations thereof associated with the user.

In an aspect, a non-transitory computer-readable medium storing program instructions for ergonomic cognitive strain management, the program instructions, when executed, perform steps of collecting sensor data associated with posture conditions, neck positioning conditions, attention features, and interaction conditions associated with a user, generating synchronized posture features and attention features by processing the sensor data, determining availability of previously stored user specific baseline information corresponding to the user, retrieving a previously stored user specific baseline when the previously stored user specific baseline information is available or determining a user specific baseline when the previously stored user specific baseline information is unavailable, determining an ergonomic cognitive strain state using the synchronized posture features, the attention features, and the user specific baseline, and generating a feedback cue based on the ergonomic cognitive strain state and the user specific baseline for outputting at the smart eyewear system.

Other aspects and advantages of the invention will become apparent from the following description, taken in conjunction with the accompanying drawings, illustrating by way of example the principles of the invention.

### BRIEF DESCRIPTION OF THE DRAWINGS

The accompanying drawings constitute a part of the description and are used to provide further understanding of the present disclosure. The drawings illustrate exemplary embodiments of the present disclosure and, together with the description, serve to explain the principles of the present disclosure.

FIG. 1 illustrates a working environment of a smart eyewear system for ergonomic cognitive strain management associated with a user, in accordance with an embodiment of the present disclosure.

FIG. 1A illustrates a schematic representation of sensor placement regions within the smart eyewear system, in accordance with an embodiment of the present disclosure.

FIG. 2 illustrates a block diagram representing components associated with the smart eyewear system and a user device, in accordance with an embodiment of the present disclosure.

FIG. 3 illustrates a functional architecture of one or more modules associated with the smart eyewear system, in accordance with an embodiment of the present disclosure.

FIG. 4 illustrates a method flow diagram representing a method for ergonomic cognitive strain management using the smart eyewear system, in accordance with an embodiment of the present disclosure.

A more complete understanding of the present disclosure and its embodiments thereof may be acquired by referring to the following description and the accompanying drawings.

### DETAILED DESCRIPTION OF THE INVENTION

Exemplary embodiments now will be described with reference to the accompanying drawings. The disclosure may, however, be embodied in many different forms and should not be construed as limited to the embodiments set forth herein; rather, these embodiments are provided so that this disclosure will be thorough and complete, and will fully convey its scope to those skilled in the art. The terminology used in the detailed description of the particular exemplary embodiments illustrated in the accompanying drawings is not intended to be limiting. In the drawings, like numbers refer to like elements.

It is to be noted, however, that the reference numerals used herein illustrate only typical embodiments of the present subject matter, and are therefore, not to be considered for limiting its scope, for the subject matter may admit to other equally effective embodiments.

The specification may refer to “an”, “another”, “one” or “some” embodiment(s) in several locations.

This does not necessarily imply that each such reference is to the same embodiment(s), or that the feature only applies to a single embodiment. Single features of different embodiments may also be combined to provide other embodiments.

As used herein, the singular forms “a”, “an” and “the” are intended to include the plural forms as well, unless expressly stated otherwise. It will be further understood that the terms “include”, “comprises”, “including” and/or “comprising” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. Furthermore, “connected” or “coupled” as used herein may include operatively connected or coupled. As used herein, the term “and/or” includes any and all combinations and arrangements of one or more of the associated listed items.

Unless otherwise defined, all terms (including technical and scientific terms) used herein have the same meaning as commonly understood by one of ordinary skills in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The detailed description includes specific details for the purpose of providing a thorough understanding of the present disclosure. However, it will be apparent to those skilled in the art that the present disclosure may be practiced without these specific details.

The present disclosure provides a smart eyewear system configured for real-time posture correction, focus monitoring, and ergonomic cognitive strain management during prolonged desk based activities, wherein the desk based activities may include at least computer usage, studying, gaming, reading, and prolonged screen interaction activities. The smart eyewear system is configured to monitor neck positioning, head orientation, posture related strain, cognitive strain, gaze behavior, attention characteristics, blue light exposure, and user engagement patterns using multimodal sensing and adaptive analysis techniques. The smart eyewear system is further configured to generate real-time feedback cues and adapt user specific behavioral baselines over time to improve ergonomic wellness and cognitive performance associated with prolonged screen based activities.

In an embodiment, the smart eyewear system comprises an eyewear frame, at least one inertial measurement unit (IMU), a plurality of pressure sensors positioned at eyewear user contact regions including a nose bridge region and/or ear contact portions, a gaze or attention monitoring sensor, and a controller communicatively coupled with the sensing modules.

The controller is configured to acquire the sensor data from the plurality of sensors and combine them to form multimodal sensor data associated with posture conditions and attention behavior, generate synchronized posture and attention features, fuse pressure features, inertial features, gaze features, blue light exposure related features, and attention features to determine an ergonomic cognitive strain state associated with a user, trigger an eyewear integrated feedback cue based on the ergonomic cognitive strain state, and adapt a user specific baseline over time according to monitored behavioral patterns and ergonomic conditions. In some embodiments, the ergonomic cognitive strain state may represent one or more ergonomic cognitive strain conditions associated with the user, including posture related strain conditions, fatigue conditions, attention reduction conditions, neck strain conditions, eye strain conditions, cognitive workload conditions, or combinations thereof.

In some embodiments, the controller may process posture related data, neck positioning related data, attention related data, interaction related data, pressure related data, inertial data, gaze related data, or combinations thereof associated with the user to determine ergonomic cognitive strain characteristics corresponding to measurable ergonomic and cognitive parameters associated with the user. The controller may further evaluate the ergonomic cognitive strain characteristics to determine an ergonomic cognitive strain state associated with the user, wherein the ergonomic cognitive strain state may represent an inferred ergonomic and cognitive status of the user corresponding to posture related strain conditions, neck strain conditions, attention degradation conditions, fatigue conditions, cognitive overload conditions, prolonged improper posture conditions, ergonomic cognitive strain conditions, or combinations thereof associated with the user.

In some embodiments, the ergonomic cognitive strain conditions may correspond to one or more detected, inferred, predicted, classified, or monitored ergonomic and cognitive conditions associated with the user, including cumulative posture degradation conditions, sustained neck misalignment conditions, repetitive movement strain conditions, reduced attentiveness conditions, cognitive fatigue conditions, elevated cognitive workload conditions, stress related conditions, distraction conditions, reduced interaction efficiency conditions, prolonged sedentary conditions, or combinations thereof. The ergonomic cognitive strain conditions may further correspond to transient conditions, persistent conditions, progressive conditions, threshold exceeding conditions, user specific deviation conditions, context dependent conditions, or combinations thereof associated with ergonomic and cognitive performance of the user.

In some embodiments, the ergonomic cognitive strain characteristics may further be utilized during threshold adaptation operations, user specific baseline adaptation operations, ergonomic cognitive strain determination operations, ergonomic cognitive strain condition determination operations, or combinations thereof associated with the user.

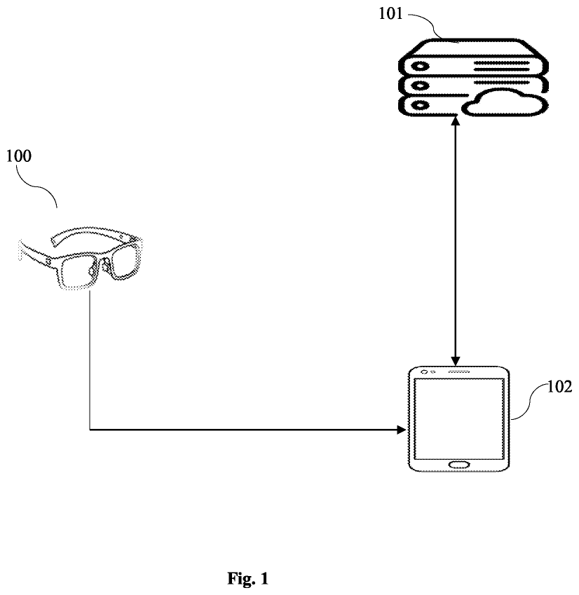

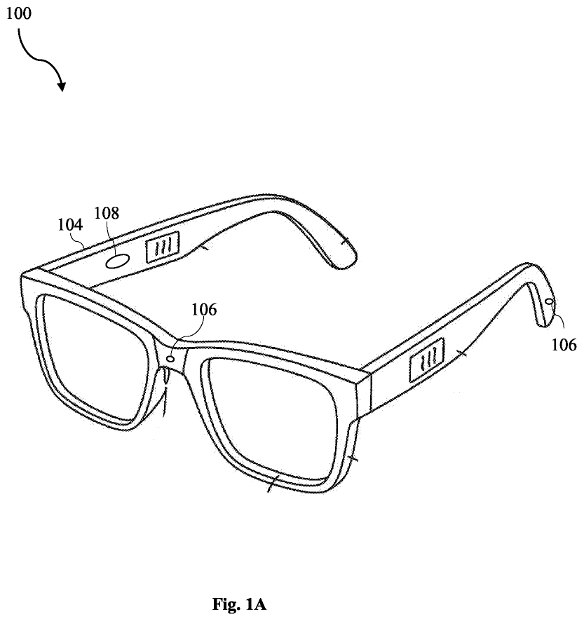

FIG. 1 illustrates a working environment of a smart eyewear system 100 for ergonomic cognitive strain management associated with a user, in accordance with an embodiment of the present disclosure. The working environment may comprise the smart eyewear system 100, a user device 102, and a server 101, which may be operatively interconnected through one or more wireless communication protocols.

In one embodiment, the smart eyewear system 100 may be implemented as smart eyewear configured to be worn by the user during prolonged desk based activities including computer usage, studying, gaming, or other screen interaction activities. However, it should be understood that the smart eyewear system 100 is not limited to a particular eyewear configuration and may alternatively be implemented as prescription eyewear, smart glasses, protective eyewear, augmented reality eyewear, or other wearable eyewear platforms configured to perform ergonomic cognitive strain management operations. In one embodiment, the smart eyewear system 100 may alternatively be implemented as a headband, visor, neckband, or other wearable platform configured to perform ergonomic cognitive strain management operations.

Unlike conventional eye health monitoring systems that primarily monitor visual fatigue or eye activity conditions, the smart eyewear system 100 disclosed herein is configured to determine ergonomic cognitive strain conditions using multimodal sensor data associated with posture characteristics, neck positioning characteristics, pressure distribution characteristics, attention characteristics, and blue light exposure conditions associated with the user.

In an embodiment, the smart eyewear system 100 may comprise a plurality of sensors configured to generate sensor data associated with posture conditions, neck positioning conditions, attention characteristics, cognitive strain conditions, blue light exposure conditions, and user interaction conditions associated with the user. The plurality of sensors may comprise at least one inertial sensing sensor configured to generate inertial data associated with head orientation, neck positioning, head tilt, and posture related movement conditions.

In some embodiments, the interaction conditions associated with the user may comprise screen interaction conditions, device usage conditions, application interaction conditions, user activity conditions, session duration conditions, keyboard interaction conditions, mouse interaction conditions, viewing behavior conditions, reading activity conditions, user engagement conditions, device proximity conditions, work intensity conditions, user response conditions, or combinations thereof associated with the user. The interaction conditions may further correspond to user behavior associated with electronic device usage, screen engagement patterns, user activity duration, application switching behavior, input activity frequency, inactivity periods, or other interaction characteristics associated with prolonged desk based activities performed by the user.

The plurality of sensors may further comprise a plurality of pressure sensors positioned at eyewear user contact regions including a nose bridge region and/or ear contact portions associated with the smart eyewear system 100. The plurality of sensors may further comprise at least one gaze or attention monitoring sensor and at least one blue light exposure monitoring sensor configured to monitor attention characteristics and prolonged screen interaction activities, such activities related to the blue light exposure related data associated with the user. In one embodiment, the blue light exposure related data may broadly comprise screen light intensity data, wavelength related exposure metrics, ambient lighting data, exposure duration information, screen interaction characteristics, environmental illumination characteristics, and derived blue light exposure conditions associated with the user. The derived blue light exposure conditions may represent cumulative, instantaneous, or behavior associated blue light exposure conditions determined using one or more of screen interaction activities, gaze behavior characteristics, environmental lighting conditions, exposure duration characteristics, and user interaction conditions associated with the user. In some embodiments, the blue light exposure monitoring sensor may include one or more photodiodes and optical filters configured to detect blue light associated with one or more predefined wavelength ranges. The eyewear device may further include an ambient light sensor configured to distinguish ambient illumination from display-generated light exposure and a display-proximity detection module configured to determine proximity of an electronic display device relative to the user. A processing module may process sensed light intensity, wavelength-range sensor data, exposure duration, and proximity information to calculate cumulative blue light exposure estimates and exposure-duration values associated with one or more monitoring periods.

In an embodiment, the smart eyewear system 100 may further comprise a controller (may be implemented using a processor) communicatively coupled with a memory and the plurality of sensors.

The controller may be configured to acquire and process the sensor data generated by the plurality of sensors, generate synchronized posture features and attention features using the sensor data, and determine an ergonomic cognitive strain state associated with the user using the synchronized posture features and the attention features, wherein the attention features may include gaze, blink, screen engagement, cognitive attention and cognitive fatigue features. The controller may be further configured to adapt a user specific baseline according to historical user associated characteristics and generate a feedback cue based on the ergonomic cognitive strain state and the user specific baseline.

In an embodiment, the smart eyewear system 100 may be operatively coupled with the user device 102 through a communication module (also referred to as wireless module) configured to support wireless communication protocols including Bluetooth, Wi Fi, Near Field Communication (NFC), or cellular communication. The user device 102 may comprise a smartphone, tablet, laptop, desktop computer, or other computing device configured to receive user associated information from the smart eyewear system 100. The user associated information may comprise posture related information, cognitive strain information, attention related information, blue light exposure related information, and historical behavioral information associated with the user.

In an embodiment, the smart eyewear system 100 may be further operatively coupled with the server 101 either directly through the wireless module or indirectly through the user device 102. The server 101 may be configured to store and process information acquired and generated by the smart eyewear system 100. In one embodiment, the server 101 may further perform processing operations associated with synchronized posture and attention feature generation, ergonomic cognitive strain determination, user specific baseline adaptation, behavioral pattern analysis, and personalized feedback generation.

FIG. 1A illustrates a schematic representation of sensor placement regions within the smart eyewear system 100, in accordance with an embodiment of the present disclosure. The smart eyewear system 100 may comprise an eyewear frame 104 configured to support a plurality of sensors associated with ergonomic cognitive strain monitoring operations. The eyewear frame 104 may be configured to be worn by a user during prolonged desk based activities including computer usage, studying, gaming, reading, or other prolonged screen interaction activities. In one embodiment, the eyewear frame 104 may be implemented as prescription eyewear, smart glasses, protective eyewear, augmented reality eyewear, or other wearable eyewear platforms configured to support sensing, processing, and feedback generation operations.

In an embodiment, the smart eyewear system 100 may comprise at least one inertial sensing sensor 108 positioned within the eyewear frame 104. The inertial sensing sensor 108 may comprise at least one accelerometer, gyroscope, magnetometer, inertial measurement unit (IMU), or combinations thereof configured to generate inertial data associated with head orientation, neck positioning, head tilt, posture related movement conditions, and user motion characteristics. The inertial sensing sensor 108 may continuously monitor positional variations associated with the head and neck region of the user to facilitate ergonomic posture monitoring and ergonomic cognitive strain determination operations.

In an embodiment, the smart eyewear system 100 may further comprise a plurality of pressure sensors 106 positioned at eyewear user contact regions associated with the eyewear frame 104. The plurality of pressure sensors 106 may be positioned at a nose bridge region and/or ear contact portions associated with the eyewear frame 104. The plurality of pressure sensors 106 may be configured to generate pressure data associated with posture induced loading conditions, pressure distribution characteristics, ergonomic strain conditions, and prolonged wear conditions associated with the user. In some embodiments, the pressure data generated by the plurality of pressure sensors 106 may include contact pressure values, pressure magnitude values, pressure distribution values, pressure differential values, temporal pressure variation values, asymmetric loading values, localized pressure concentration values, dynamic pressure change values, pressure imbalance values, or combinations thereof associated with the user. The pressure data may further correspond to posture related loading conditions, head orientation conditions, neck positioning conditions, ergonomic strain conditions, posture imbalance conditions, or prolonged improper posture conditions associated with the user. The plurality of pressure sensors 106 may further facilitate determination of asymmetric loading conditions, posture imbalance conditions, and neck positioning conditions associated with the user.

In some embodiments, the pressure sensors 106 positioned at nose bridge contact regions and ear-contact portions of the frame to generate pressure measurement data corresponding to contact forces between the eyewear system 100 and the user. In some embodiments, the pressure sensors 106 may be positioned at left-side nose contact regions, right-side nose contact regions, nose bridge regions, ear contact portions, or combinations thereof associated with the eyewear frame 104. A controller, implemented using processor, processing unit or combination thereof, may process pressure values from multiple sensing locations to generate pressure distribution data representing spatial distribution of pressure across the frame. The pressure distribution data may be determined based on relative pressure magnitudes corresponding to comparative pressure levels detected at different sensing locations, pressure differentials between sensing locations corresponding to differences in measured pressure values between left-side and right-side frame portions, temporal pressure variations corresponding to changes in pressure values over time, or combinations thereof.

For example, greater pressure detected at a left nose-contact region relative to a right nose-contact region may indicate head tilt or lateral imbalance Similarly, greater pressure detected at a right nose contact region relative to a left nose contact region may indicate opposite directional head tilt conditions or lateral imbalance conditions associated with the user. Further, increased pressure at the nose bridge region combined with reduced pressure at ear-contact portions may indicate forward head posture, neck inclination, or slouching. In some embodiments, the controller may further compare pressure distribution data over time to determine posture shifts, head orientation changes, or prolonged improper posture conditions during sitting, walking, or electronic device usage.

In one embodiment, the smart eyewear system 100 may further comprise at least one gaze or attention monitoring sensor (not illustrated) positioned within the eyewear frame 104 and configured to monitor gaze behavior, screen engagement patterns, eye movement characteristics, blink characteristics, and attention characteristics associated with the user. In another embodiment, the smart eyewear system 100 may further comprise at least one blue light exposure monitoring sensor configured to monitor blue light exposure conditions associated with prolonged screen interaction activities performed by the user.

The gaze or attention monitoring sensor and the blue light exposure monitoring sensor may cooperate with the inertial sensing sensor 108 and the plurality of pressure sensors 106 to generate multimodal sensor data associated with posture conditions, cognitive strain conditions, and user interaction conditions associated with the user.

In one embodiment, the gaze or attention monitoring sensor may include an inward-facing camera, an eye-facing optical sensor, or a forward-facing camera configured to infer screen engagement patterns, gaze directions conditions, or attention characteristics associated with the user.

In an embodiment, the smart eyewear system 100 may further comprise a controller communicatively coupled with the inertial sensing sensor 108, the plurality of pressure sensors 106, the gaze or attention monitoring sensor, and the blue light exposure monitoring sensor. The controller may be configured to acquire and process sensor data generated by the plurality of sensors and generate synchronized posture features and attention features using the sensor data.

The controller may be further configured to fuse the synchronized posture features and the attention features to determine an ergonomic cognitive strain state associated with the user. The ergonomic cognitive strain state may comprise at least one of a posture related strain condition, a neck positioning condition, a cognitive strain condition, an attention degradation condition, or a blue light exposure condition associated with the user.

In one embodiment, the controller may be further configured to adapt a user specific baseline according to historical posture characteristics, attention characteristics, cognitive strain characteristics, behavioral patterns, and user interaction conditions associated with the user. The controller may further generate a feedback cue based on the ergonomic cognitive strain state and the user specific baseline associated with the user. The feedback cue may comprise at least one of a haptic feedback cue, a visual feedback cue, or an audio feedback cue configured to provide a real-time corrective notification associated with the ergonomic cognitive strain state.

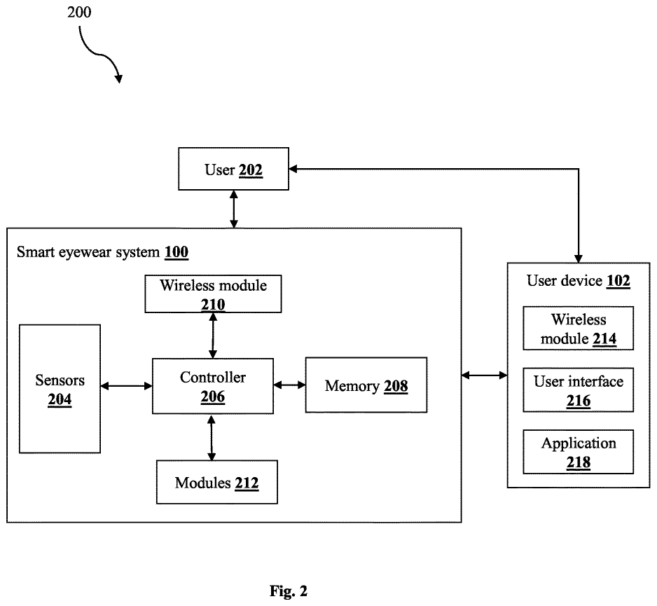

FIG. 2 illustrates a block diagram 200 representing components associated with the smart eyewear system 100 and a user device 102, in accordance with an embodiment of the present disclosure. In one embodiment, the smart eyewear system 100 may communicate user associated information and ergonomic cognitive strain information associated with a user 202 to the user device 102 through one or more wireless communication protocols using wireless modules (discussed later). Further, the user device 102 may transmit user associated configuration settings, personalized feedback settings, operational instructions, synchronization instructions, and user interaction information to the smart eyewear system 100 for adaptive ergonomic cognitive strain management operations.

In an embodiment, the smart eyewear system 100 may comprise a plurality of sensors 204, a controller 206, a memory 208, a wireless module 210, and one or more modules 212 configured to perform ergonomic cognitive strain management operations. The plurality of sensors 204 may receive positional conditions, movement, pressure, gaze, environmental lighting, cognitive activity, and user interaction conditions associated with the user 202 as input and may generate corresponding sensor data as output. The plurality of sensors 204 may comprise at least one inertial sensing sensor configured to generate inertial data associated with head orientation, neck positioning, head tilt, posture related movement conditions, and user motion characteristics associated with the user 202.

The plurality of sensors 204 may further comprise a plurality of pressure sensors positioned at eyewear user contact regions including a nose bridge region and/or ear contact portions associated with the smart eyewear system 100. The plurality of pressure sensors may generate pressure data associated with posture induced loading conditions, pressure distribution characteristics, ergonomic strain conditions, prolonged wear conditions, asymmetric loading conditions, and posture imbalance conditions associated with the user 202

In one embodiment, the plurality of sensors 204 may further comprise at least one gaze or attention monitoring sensor configured to receive eye movement conditions, gaze direction conditions, blink conditions, and screen engagement conditions associated with the user 202 as input and generate gaze related data and attention related data as output. The plurality of sensors 204 may further comprise at least one blue light exposure monitoring sensor configured to receive screen illumination conditions and environmental lighting conditions as input and generate blue light exposure related data associated with prolonged screen interaction activities associated with the user 202 as output.

In another embodiment, the plurality of sensors 204 may further comprise at least one ambient light sensing sensor configured to receive environmental lighting conditions as input and generate environmental lighting data associated with a surrounding environment of the user 202 as output. In another embodiment, the plurality of sensors 204 may further comprise at least one electroencephalography (EEG) sensing sensor configured to receive neurological activity conditions associated with the user 202 as input and generate cognitive activity related data as output.

In an embodiment, the plurality of sensors 204 may continuously generate sensor data associated with posture conditions, neck positioning conditions, cognitive strain conditions, attention characteristics, blue light exposure conditions, and user interaction conditions associated with the user 202. The sensor data generated by the plurality of sensors 204 may be transmitted to the controller 206 for ergonomic cognitive strain management operations. In an embodiment, the controller 206 may be communicatively coupled with the plurality of sensors 204, the memory 208, the wireless module 210, and the one or more modules 212.

The controller 206 may receive the sensor data generated by the plurality of sensors 204 as input and may generate synchronized posture features, attention features, ergonomic cognitive strain states, user specific baseline information, and feedback control instructions as output.

The controller 206 may acquire and process the sensor data generated by the plurality of sensors 204 and may generate synchronized posture features and attention features using the sensor data. The controller 206 may further temporally align and fuse the synchronized posture features and the attention features to determine an ergonomic cognitive strain state associated with the user 202. The ergonomic cognitive strain state may comprise at least one of a posture related strain condition, a neck positioning condition, a cognitive strain condition, an attention degradation condition, or a blue light exposure condition associated with the user 202. In some embodiments, the controller 206 may determine whether previously stored user specific baseline information corresponding to the user 202 is available in the memory 208. The user specific baseline information may correspond to previously generated posture profiles, attention profiles, interaction profiles, ergonomic monitoring profiles, or combinations thereof associated with the user. In response to determining that the previously stored user specific baseline information is available, the controller 206 may retrieve a previously stored user specific baseline associated with the user from the memory 208 for subsequent ergonomic cognitive strain analysis. In response to determining that the previously stored user specific baseline information is unavailable, the controller 206 may determine a user specific baseline using sensor data associated with posture conditions, neck positioning conditions, attention characteristics, user interaction conditions, synchronized posture features, attention features, or combinations thereof associated with the user. In some embodiments, the determined or retrieved user specific baseline may be utilized for personalized ergonomic cognitive strain assessment and feedback generation associated with the user 202.

In an embodiment, the controller 206 may further adapt the user specific baseline according to historical posture characteristics, attention characteristics, cognitive strain characteristics, behavioral patterns, and user interaction conditions associated with the user 202. In some embodiments, the controller 206 may update the user-specific baseline by dynamically adjusting one or more threshold values associated with posture characteristics, attention characteristics, or ergonomic cognitive strain characteristics of the user. The threshold values may correspond to posture deviation limits, attention variation limits, cognitive strain indicators, movement frequency limits, gaze fixation durations, blink rate ranges, head orientation ranges, shoulder alignment ranges, or combinations thereof.

In some embodiments, the controller 206 may establish one or more threshold values during a calibration period using sensor data corresponding to normal user behavior or preferred ergonomic conditions associated with the user 202. The calibration period may correspond to an initial monitoring duration during which the smart eyewear system collects posture related data, neck positioning related data, attention related data, interaction related data, or combinations thereof associated with the user under normal operating conditions. In some embodiments, the controller 206 may utilize the collected sensor data to determine user specific threshold values corresponding to posture characteristics, ergonomic cognitive strain characteristics, attention characteristics, or combinations thereof associated with the user. The established threshold values may subsequently be utilized during ergonomic cognitive strain determination operations and threshold adaptation operations associated with the user 202.

In some embodiments, the controller may dynamically adjust one or more threshold values according to confidence scores associated with reliability characteristics of the sensor data. The confidence scores may correspond to measurement consistency, signal stability, temporal continuity, sensor accuracy, environmental interference characteristics, inconsistent movement characteristics, or combinations thereof associated with the sensor data. In some embodiments, sensor data having low confidence scores, inconsistent measurements, transient anomalies, or unreliable sensing characteristics may be excluded or assigned reduced weighting during threshold adaptation operations. The controller may thereby reduce influence of unreliable sensor measurements and improve reliability of ergonomic cognitive strain determination operations associated with the user.

In some embodiments, the controller 206 may initially establish the threshold values during a calibration period in which sensor data corresponding to normal or preferred user behavior is collected over a predetermined duration. The controller 206 may generate baseline statistical parameters including average values, deviation ranges, variance values, trend characteristics, or confidence intervals associated with the collected sensor data.

In some embodiments, the controller 206 may continuously receive updated sensor data from one or more sensors and compare the updated sensor data with the established baseline statistical parameters. The controller 206 may determine whether recurring deviations, sustained behavioral changes, or gradual ergonomic adaptations are present over a predefined monitoring interval. In response to determining that the recurring deviations satisfy one or more adaptation criteria, the controller 206 may automatically modify the one or more threshold values. For example, the controller 206 may increase or decrease a posture deviation threshold according to a moving average of detected posture angles, adjust an attention variation threshold according to measured gaze stability over time, or modify a cognitive strain threshold according to detected fatigue indicators accumulated across multiple monitoring sessions.

In some embodiments, the controller 206 may perform the adjustment using weighted historical data, rolling time windows, adaptive filtering techniques, machine learning models, statistical regression models, or combinations thereof. More recent sensor data may be assigned greater weighting relative to older sensor data such that the threshold values progressively adapt to current user behavior while limiting abrupt fluctuations. In some embodiments, the controller 206 may further segment the sensor data according to contextual conditions including time of day, application type, task category, seating configuration, device orientation, or environmental conditions, and may independently adjust threshold values corresponding to each contextual condition.

In some embodiments, the controller 206 may further determine adjustment rates or adjustment magnitudes according to confidence scores associated with the sensed data. Sensor readings having low confidence values, inconsistent measurements, or transient anomalies may be excluded or assigned reduced weighting during threshold adaptation. The dynamic adjustment of the one or more threshold values may thereby enable personalized monitoring, gradual adaptation to evolving user behavior, reduction of false positive ergonomic alerts, and improved detection accuracy corresponding to changing ergonomic and cognitive conditions over time.

The controller 206 may further generate a feedback cue based on the ergonomic cognitive strain state and the user specific baseline associated with the user 202. The feedback cue may comprise at least one of a haptic feedback cue, a visual feedback cue, or an audio feedback cue configured to provide a real-time corrective notification associated with the ergonomic cognitive strain state. In one embodiment, the controller 206 may further perform on device processing of the sensor data within the smart eyewear system 100 to facilitate real-time ergonomic cognitive strain determination operations.

In one embodiment, the plurality of sensors 204 may further comprise one or more electroencephalography (EEG) electrodes positioned at temple-contact regions, forehead-contact regions, ear-contact regions, or combinations thereof associated with the eyewear frame 104. The one or more EEG electrodes may be configured to receive neurological activity conditions associated with the user as input and generate EEG data associated with cognitive activity conditions, cognitive attention conditions, cognitive fatigue conditions, mental workload conditions, and focus characteristics associated with the user as output. Further, the controller 206 and/or the one or more data acquisition and processing modules may process the EEG data to generate cognitive activity features associated with cognitive strain determination operations, attention evaluation operations, behavioral analysis operations, and ergonomic-cognitive strain management operations associated with the user.

In an embodiment, the memory 208 may be communicatively coupled with the controller 206. The memory 208 may receive sensor data, posture characteristics, attention characteristics, cognitive strain characteristics, historical behavioral information, user interaction conditions, synchronized posture features, attention features, ergonomic cognitive strain states, user specific baseline information, and executable instructions as input for storage operations. Further, the memory 208 and may provide stored executable instructions, historical behavioral information, synchronized posture features, attention features, ergonomic cognitive strain states, and user specific baseline information as output to the controller 206 for ergonomic cognitive strain determination operations, adaptive baseline updating operations, behavioral analysis operations, and feedback generation operations. The memory 208 may comprise volatile memory, nonvolatile memory, Random Access Memory (RAM), Read Only Memory (ROM), flash memory, or combinations thereof. In an embodiment, the wireless module 210 may facilitate bidirectional wireless communication between the smart eyewear system 100 and the user device 102. The wireless module 210 may receive posture related information, neck positioning information, cognitive strain information, attention related information, blue light exposure related information, historical behavioral information, ergonomic cognitive strain states, synchronized posture features, attention features, and feedback related information from the controller 206 as input and may transmit the received information to the user device 102 as output.

Further, the wireless module 210 may receive user specific configuration settings, operational instructions, synchronization requests, user preferences, personalized feedback settings, and application generated control instructions from the user device 102 as input and may transmit the received information to the controller 206 as output. The wireless module 210 may support one or more wireless communication protocols including Bluetooth, Bluetooth Low Energy (BLE), Wi Fi, Near Field Communication (NFC), Zigbee, cellular communication protocols, or combinations thereof.

In an embodiment, the one or more modules 212 may comprise a sensing module, a data acquisition and processing module, a fusion and evaluation module, an adaptive intelligence module, and a feedback and communication module configured to perform ergonomic cognitive strain management operations associated with the user 202.

The controller 206 may communicate sensor data, synchronized posture features, attention features, user specific baseline information, ergonomic cognitive strain states, and user interaction information to the one or more modules 212 for further processing and analysis. The one or more modules 212 may process the received information to generate behavioral analysis information, strain evaluation information, and feedback information, and may transmit the generated information back to the controller 206. The controller 206 may further determine the ergonomic cognitive strain state associated with the user 202 using the synchronized posture features, the attention features, the user specific baseline associated with the user 202, and the information generated by the one or more modules 212.

The adaptive intelligence module may further update the user specific baseline according to historical behavioral patterns and user interaction conditions associated with the user 202. The feedback and communication module may further generate real-time corrective feedback information associated with the ergonomic cognitive strain state.

In an embodiment, the user device 102 may comprise a wireless module 214 (configured to establish a wireless connection with the wireless module 210), a user interface 216, and an application 218. The wireless module 214 may facilitate bidirectional communication between the user device 102 and the smart eyewear system 100. The application 218 executing on the user device 102 may receive posture related information, neck positioning information, ergonomic cognitive strain information, attention related information, cognitive strain information, blue light exposure related information, historical behavioral information, synchronized posture features, attention features, and feedback related information from the smart eyewear system 100 through the wireless module 214 as input.

In one embodiment, the adaptive intelligence modules may implement adaptive machine learning models configured to learn user associated ergonomic patterns, attention characteristics, cognitive strain characteristics, and behavioral response patterns over time.

The application 218 may process the received information and generate graphical information, behavioral analytics information, historical trend information, personalized ergonomic recommendations, user specific configuration settings, and personalized feedback settings as output. The generated information may be displayed through the user interface 216 to facilitate ergonomic monitoring operations and cognitive strain monitoring operations associated with the user 202.

In one embodiment, the application 218 may further facilitate customization of feedback sensitivity levels, notification characteristics, ergonomic monitoring preferences, and cognitive strain management settings associated with the user 202. In one embodiment, the application 218 may further integrate with workplace productivity software platforms to facilitate context aware ergonomic coaching operations and user productivity monitoring operations.

In one embodiment, the application 218 may further generate operational instructions, synchronization instructions, behavioral preference information, and user interaction information associated with the user 202 as output. The generated information may be transmitted to the smart eyewear system 100 through the wireless module 214 to facilitate adaptive ergonomic cognitive strain management operations. The application 218 may further facilitate historical trend visualization, behavioral analytics, personalized ergonomic monitoring operations, and user associated ergonomic cognitive strain management operations associated with the user 202.

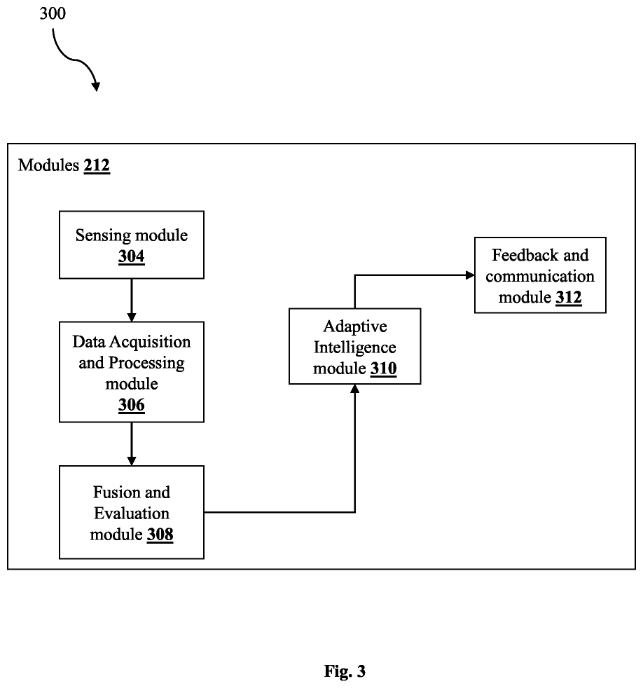

FIG. 3 illustrates a functional architecture 300 of one or more modules 212 associated with the smart eyewear system 100, in accordance with an embodiment of the present disclosure. The one or more modules 212 may comprise a sensing module 304, one or more data acquisition and processing modules 306, a fusion and evaluation module 308, one or more adaptive intelligence modules 310, and a feedback and communication module 312 configured to perform ergonomic cognitive strain management operations associated with a user.

The one or more modules 212 may be communicatively coupled with the controller 206, the plurality of sensors 204, the memory 208, and the wireless module 210 associated with the smart eyewear system 100. In one embodiment, the one or more modules 212 may facilitate a processing pipeline comprising sensing operations, synchronized feature generation operations, fusion operations of the multimodal sensor data, ergonomic cognitive strain determination operations, adaptive baseline learning operations, and feedback generation operations associated with the user.

In an embodiment, the sensing module 304 may receive positional conditions, movement conditions, pressure conditions, gaze conditions, attention conditions, environmental lighting conditions, blue light exposure conditions, cognitive activity conditions, and user interaction conditions associated with the user as input from the plurality of sensors 204.

The sensing module 304 may process the received input and generate sensor data associated with posture conditions, neck positioning conditions, posture induced loading conditions, cognitive strain conditions, cognitive fatigue conditions, gaze behavior, attention characteristics, blue light exposure conditions, and user interaction conditions as output.

The sensing module 304 may further transmit the generated sensor data to the one or more data acquisition and processing modules 306. Further, the sensing module 304 may receive synchronization instructions, calibration instructions, activation instructions, and sensor management instructions from the controller 206 to facilitate synchronized sensing operations associated with the plurality of sensors 204.

In an embodiment, the one or more data acquisition and processing modules 306 may receive sensor data generated by the sensing module 304 as input. The data acquisition and processing module 306 may acquire, organize, filter, normalize, temporally align, and process the sensor data to generate synchronized posture features and attention features associated with the user as output. The synchronized posture features may comprise head orientation features, neck positioning features, head tilt features, posture related movement features, posture induced loading features, asymmetric loading features, and pressure distribution features associated with the user.

The attention features may comprise gaze behavior features, eye movement features, blink characteristics, screen engagement features, blue light exposure features, cognitive attention features, and cognitive fatigue features associated with the user. The one or more data acquisition and processing modules 306 may further transmit the synchronized posture features and the attention features to the fusion and evaluation module 308.

In an embodiment, the fusion and evaluation module 308 may receive synchronized posture features and attention features generated by the data acquisition and processing module 306 as input. The fusion and evaluation module 308 may temporally align and fuse the synchronized posture features and the attention features to generate fused multimodal ergonomic cognitive sensing features associated with the user as output. The fused multimodal ergonomic cognitive sensing features may comprise fused posture features, fused pressure features, fused gaze features, fused blue light exposure features, fused attention features, and fused cognitive strain features associated with the user 202.

The fusion and evaluation module 308 may further determine an ergonomic cognitive strain state associated with the user 202 using the fused multimodal ergonomic cognitive sensing features. The ergonomic cognitive strain state may comprise at least one of a posture related strain condition, a neck positioning condition, a cognitive strain condition, a cognitive fatigue condition, an attention degradation condition, a posture imbalance condition, or a blue light exposure condition associated with the user.

In one embodiment, the fusion and evaluation module 308 may further determine neck positioning conditions using inertial sensing features and pressure distribution characteristics associated with eyewear user contact regions of the smart eyewear system 100. The fusion and evaluation module 308 may further determine ergonomic strain conditions and cognitive strain conditions using fused multimodal ergonomic cognitive sensing features associated with posture characteristics, attention characteristics, pressure distribution characteristics, gaze behavior characteristics, and blue light exposure characteristics associated with the user.

In one embodiment, the fusion and evaluation module 308 may further detect slouching conditions, head droop conditions, gaze disengagement conditions, and posture deviation conditions associated with the user 202 using the fused multimodal ergonomic cognitive sensing features. The fusion and evaluation module 308 may further fuse pressure features, inertial sensing features, gaze features, and attention features to determine the ergonomic cognitive strain state associated with the user.

Further, the fusion and evaluation module 308 may transmit the ergonomic cognitive strain state, fused multimodal ergonomic cognitive sensing features, synchronized posture features, attention features, and strain evaluation information to the adaptive intelligence module 310. The fusion and evaluation module 308 may further receive historical behavioral information, adaptive baseline information, threshold adaptation information, and behavioral analysis information from the adaptive intelligence module 310 to facilitate adaptive ergonomic cognitive strain determination operations. The adaptive intelligence module 310 may further perform longitudinal behavioral analysis operations to identify long term ergonomic trends, attention trends, cognitive fatigue trends, and user wellness patterns associated with the user.

In an embodiment, the adaptive intelligence module 310 may receive ergonomic cognitive strain states, fused multimodal ergonomic cognitive sensing features, synchronized posture features, attention features, strain evaluation information, historical behavioral information, and user interaction information as input. The adaptive intelligence module 310 may analyze the received information to generate adaptive baseline information, behavioral analysis information, personalized ergonomic information, user associated strain evaluation information, and feedback generation information as output.

The adaptive intelligence module 310 may further adapt a user specific baseline according to historical posture characteristics, attention characteristics, cognitive strain characteristics, cognitive fatigue characteristics, behavioral patterns, and user interaction conditions associated with the user. Further, the adaptive intelligence module 310 may transmit adaptive baseline information, behavioral analysis information, personalized ergonomic information, synchronized feature adaptation information, and feedback generation information to the feedback and communication module 312.

The adaptive intelligence module 310 may further receive feedback response information, user preference information, user interaction information, and communication control information from the feedback and communication module 312 to facilitate adaptive learning operations and personalized ergonomic cognitive strain management operations.

In an embodiment, the feedback and communication module 312 may receive adaptive baseline information, behavioral analysis information, personalized ergonomic information, ergonomic cognitive strain states, fused multimodal ergonomic cognitive sensing features, and feedback generation information from the adaptive intelligence module 310 as input.

The feedback and communication module 312 may process the received information to generate feedback cues, communication information, user notifications, ergonomic recommendations, corrective posture instructions, cognitive strain alerts, cognitive fatigue alerts, and user associated monitoring information as output. The feedback cues may comprise at least one of haptic feedback cues, visual feedback cues, or audio feedback cues configured to provide real-time corrective notifications associated with ergonomic cognitive strain conditions of the user. The feedback cues may further facilitate proactive ergonomic coaching operations and real-time behavioral correction operations during ongoing desk based activities associated with the user. In some embodiments, the monitoring information communicated to the user device 102 may comprise posture related information, ergonomic cognitive strain information, attention related information, blue light exposure related information, and historical behavioral information associated with the user.

The posture related information may include posture deviation data, head orientation data, neck inclination data, or posture trend information. The ergonomic cognitive strain information may include fatigue indicators, strain scores, cognitive workload indicators, or prolonged strain conditions detected using multimodal sensor data. The attention related information may include gaze stability information, blink pattern information, attention duration information, or distraction related events.

The blue light exposure related information may include cumulative exposure values, exposure duration values, wavelength related exposure conditions, or exposure trend information associated with electronic device usage. In some embodiments, the historical behavioral information may include historical posture patterns, attention variation trends, ergonomic behavior summaries, or long-term usage characteristics associated with the user.

The monitoring information may be communicated to the user device 102 for presentation through graphical interfaces, alerts, dashboards, summaries, recommendations, or historical analytics.

The feedback and communication module 312 may further transmit the generated communication information, ergonomic cognitive strain information, historical behavioral information, fused multimodal ergonomic cognitive sensing features, synchronized posture features, attention features, and user associated ergonomic information to the wireless module 210 and the user device 102 via controller 206, for visualization and monitoring operations.

Further, the feedback and communication module 312 may receive user associated configuration settings, personalized feedback settings, operational instructions, synchronization instructions, user preferences, and behavioral response information from the user device 102 through the wireless module 210 to facilitate adaptive feedback generation operations and personalized ergonomic cognitive strain management operations.

In one embodiment, the one or more modules 212 may continuously perform ergonomic posture monitoring operations and cognitive strain monitoring operations during prolonged desk-based activities including computer usage, studying, gaming, reading, or prolonged screen interaction activities associated with the user. In another embodiment, the one or more modules 212 may further perform on device processing operations within the smart eyewear system 100 to facilitate real-time ergonomic cognitive strain determination operations and real-time corrective feedback generation operations. In one embodiment, the on-device processing operations may reduce communication latency and improve privacy protection associated with user related sensor data and ergonomic cognitive strain information.

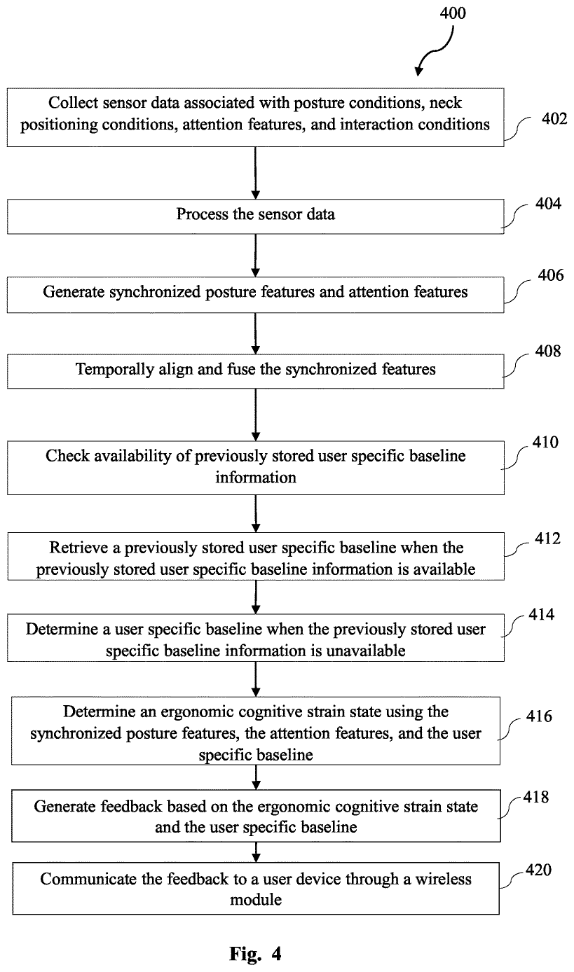

FIG. 4 illustrates a flow diagram 400 representing a method for ergonomic cognitive strain management using the smart eyewear system 100, in accordance with an embodiment of the present disclosure.

At step 402, a plurality of sensors 204 integrated into an eyewear frame 104 may collect sensor data associated with posture conditions, neck positioning conditions, attention features, and interaction conditions associated with a user 202 wearing the smart eyewear system 100. In some embodiments, the plurality of sensors 204 may comprise inertial sensors, pressure sensors, gaze monitoring sensors, ambient light sensing sensors, blue light exposure monitoring sensors, electroencephalography sensing sensors, or combinations thereof configured to monitor ergonomic and cognitive conditions associated with the user 202.

At step 404, a controller 206 may process the sensor data generated by the plurality of sensors 204. In some embodiments, the controller 206 may filter, normalize, classify, synchronize, or analyze the sensor data to facilitate ergonomic cognitive strain determination operations associated with the user 202.

At step 406, the controller 206 may generate synchronized posture features and attention features using the sensor data. The synchronized posture features may correspond to posture orientation characteristics, neck positioning characteristics, posture imbalance characteristics, head tilt characteristics, movement characteristics, or combinations thereof associated with the user 202. Further, the attention features may correspond to gaze direction characteristics, blink characteristics, attention duration characteristics, screen engagement characteristics, cognitive attention characteristics, or combinations thereof associated with the user 202.

At step 408, the controller 206 may temporally align and fuse the synchronized posture features and the attention features. In some embodiments, the controller 206 may correlate posture related characteristics and attention related characteristics occurring during corresponding monitoring intervals to facilitate ergonomic cognitive strain evaluation operations associated with the user 202.

At step 410, the controller 206 may check availability of previously stored user specific baseline information corresponding to the user 202. In some embodiments, the previously stored user specific baseline information may correspond to previously generated posture profiles, attention profiles, interaction profiles, ergonomic monitoring profiles, or combinations thereof associated with the user 202.

At step 412, the controller 206 may retrieve a previously stored user specific baseline associated with the user 202 when the previously stored user specific baseline information is available. In some embodiments, the previously stored user specific baseline may comprise previously established posture characteristics, attention characteristics, interaction characteristics, ergonomic monitoring characteristics, or combinations thereof associated with the user 202.

At step 414, the controller 206 may determine a user specific baseline associated with the user 202 when the previously stored user specific baseline information is unavailable. In some embodiments, the controller 206 may determine the user specific baseline using posture conditions, neck positioning conditions, synchronized posture features, attention features, interaction conditions, or combinations thereof associated with the user 202.

At step 416, the controller 206 may determine an ergonomic cognitive strain state associated with the user 202 using the synchronized posture features, the attention features, and the user specific baseline. In some embodiments, the ergonomic cognitive strain state may correspond to posture related strain conditions, cognitive strain conditions, neck strain conditions, attention degradation conditions, fatigue conditions, blue light exposure conditions, or combinations thereof associated with the user 202.

At step 418, the controller 206 may generate feedback based on the ergonomic cognitive strain state and the user specific baseline. In some embodiments, the feedback may comprise at least one of a visual feedback indication, an audio feedback indication, a haptic feedback indication, an ergonomic recommendation, a posture correction notification, a fatigue alert, or an attention reminder associated with the user 202.

At step 420, the controller 206 may communicate the feedback to a user device 102 through a wireless module 210. In some embodiments, the wireless module 210 may support wireless communication protocols including Bluetooth, Bluetooth Low Energy (BLE), Wi-Fi, Near Field Communication (NFC), Zigbee, cellular communication protocols, or combinations thereof.

The program instructions responsible for executing the various steps of the present invention may be stored on any suitable non-transitory computer-readable medium, which can include, without limitation, magnetic storage devices such as hard disk drives (HDDs) or floppy disks, optical storage media such as compact discs (CDs) or digital versatile discs (DVDs), solid-state memory devices including flash drives or non-volatile memory, and other forms of electronic or magnetic storage capable of retaining digital data. Such non-transitory computer-readable medium may be locally attached to a computing device or accessible via networked storage, cloud-based systems, or other distributed architectures, thereby enabling the program instructions to be retrieved and executed by one or more processors to perform the operations and functions of the invention.

In some embodiments, the memory 208 of the eyewear illustrated in FIG. 2 is one form of the non-transitory computer-readable medium. The memory 208 may store program instructions, software modules, executable code, machine-readable instructions, firmware instructions, or combinations thereof associated with ergonomic cognitive strain management operations. The program instructions may be retrieved from the memory 208 and executed by the controller 206 to perform sensor data collection operations, synchronized feature generation operations, user specific baseline determination operations, ergonomic cognitive strain state determination operations, feedback cue generation operations, adaptive threshold adjustment operations, or combinations thereof associated with the user 202.

Although implementations of the smart eyewear system for ergonomic cognitive strain management have been described in language specific to structural features and/or methods, it is to be understood that the appended claims are not necessarily limited to the specific features or methods described. Rather, the specific features and methods are disclosed as example implementations for ergonomic cognitive strain monitoring, posture correction, focus monitoring, adaptive baseline learning, and feedback generation associated with a user.

The invention has been described above with reference to numerous embodiments and specific examples. Many variations will suggest themselves to those skilled in this art in light of the above detailed description. All such obvious variations are within the full intended scope of the appended claims.

1. A smart eyewear system comprising:
a plurality of sensors integrated into an eyewear frame and configured to collect sensor data associated at least with posture conditions, neck positioning conditions, attention features, and interaction conditions, associated with a user wearing the smart eyewear system;
a controller connected with the plurality of sensors,
wherein the controller is configured to:
generate synchronized posture features and attention features by processing the sensor data;
check whether previously stored user specific baseline information corresponding to the user is available, retrieve a previously stored user specific baseline associated with the user when the previously stored user specific baseline information is available, or determine a user specific baseline associated with the user when the previously stored user specific baseline information is unavailable;
determine an ergonomic cognitive strain state associated with the user based on the synchronized posture features, the attention features, and the user specific baseline; and
generate a feedback cue based on the ergonomic cognitive strain state and the user specific baseline and output the feedback cue at the smart eyewear system.

2. The smart eyewear system as claimed in claim 1, further comprising a wireless module configured to communicate the feedback cue to a user device.

3. The smart eyewear system as claimed in claim 2, wherein the wireless module is communicatively coupled with the controller to communicate at least one of the sensor data, processed sensor data, or monitoring information to the user device.

4. The smart eyewear system as claimed in claim 3, wherein the user device communicates the at least one of the sensor data, processed sensor data, and monitoring information to a remote server periodically.

5. The smart eyewear system as claimed in claim 1, further comprising a memory communicatively coupled with the controller, wherein the memory is configured to store at least one of sensor data, processed sensor data, historical posture conditions, attention features, ergonomic cognitive strain states, behavioral patterns, user specific baseline information, or monitoring information associated with the user.

6. The smart eyewear system as claimed in claim 5, wherein the controller is configured to retrieve the user specific baseline information from the memory.

7. The smart eyewear system as claimed in claim 1, wherein the feedback cue comprises at least one of a visual indication, an audio indication, a haptic indication, a notification, an alert, a posture correction recommendation, a fatigue indication, an attention reminder, a blue light exposure warning, or a user guidance output associated with the ergonomic cognitive strain state.

8. The smart eyewear system as claimed in claim 1, wherein the controller is further configured to update the user specific baseline based on historical posture conditions, attention conditions, the ergonomic cognitive strain state, behavioral patterns, and user interaction conditions associated with the user.

9. The smart eyewear system as claimed in claim 1, wherein the plurality of sensors comprises at least one inertial sensing sensor configured to generate inertial data associated with head orientation, neck positioning, head tilt, and posture related movement conditions associated with the user, and a plurality of pressure sensors positioned at eyewear user contact regions including a nose bridge region and ear contact portions associated with the eyewear frame.

10. The smart eyewear system as claimed in claim 9, wherein the plurality of pressure sensors is configured to generate pressure data associated with posture induced loading conditions, pressure distribution characteristics, and ergonomic strain conditions associated with the user.

11. The smart eyewear system as claimed in claim 1, wherein the plurality of sensors further comprises at least one gaze or attention monitoring sensor configured to generate gaze related data and attention related data associated with gaze behavior, screen engagement patterns, and attention characteristics of the user, and at least one blue light exposure monitoring sensor configured to generate blue light exposure related data associated with prolonged screen interaction activities of the user.

12. The smart eyewear system as claimed in claim 1, wherein the plurality of sensors further comprises:
one or more pressure sensors positioned at a nose bridge region and ear contact portions associated with the eyewear frame to generate pressure data;
at least one inertial sensing sensor configured to generate inertial data associated with head orientation and the neck positioning conditions associated with the user; and
at least one gaze or attention monitoring sensor configured to generate gaze related data and attention related data associated with the user, and
wherein the controller is configured to temporally align and fuse the pressure data, the inertial data, the gaze related data and attention related data to determine an ergonomic cognitive strain state associated with the user, and to generate the feedback cue based on the ergonomic cognitive strain state and the user specific baseline.

13. The smart eyewear system as claimed in claim 1, wherein the controller is further configured to temporally align the sensor data to generate synchronized posture features and attention features and fuse the synchronized posture features and the attention features to determine the ergonomic cognitive strain state associated with the user.

14. The smart eyewear system as claimed in claim 1, wherein the ergonomic cognitive strain state comprises at least one of a posture related strain condition, a neck positioning condition, a cognitive strain condition, an attention degradation condition, or a blue light exposure condition associated with the user.

15. The smart eyewear system as claimed in claim 1, wherein the controller is further configured to adapt the user specific baseline by dynamically adjusting one or more threshold values associated with posture characteristics, attention characteristics, or ergonomic cognitive strain characteristics based on historical sensor data and user interaction conditions associated with the user.

16. The smart eyewear system as claimed in claim 1, wherein the controller is further configured to perform on device processing of the sensor data associated with the user.

17. The smart eyewear system as claimed in claim 1, wherein the plurality of sensors further comprises at least one ambient light sensor configured to generate environmental lighting data associated with a surrounding environment of the user.

18. The smart eyewear system as claimed in claim 1, wherein the plurality of sensors further comprises at least one electroencephalography (EEG) sensing sensor configured to generate cognitive activity related data associated with the user.

19. The smart eyewear system as claimed in claim 12, wherein the pressure data comprises pressure distribution data and pressure differential data associated with the one or more pressure sensors, and wherein the controller is further configured to determine posture imbalance conditions and neck positioning conditions using the pressure distribution data and the pressure differential data, wherein greater pressure at a left nose contact region relative to a right nose contact region or greater pressure at the right nose contact region relative to the left nose contact region indicates head tilt conditions or lateral imbalance conditions, and increased pressure at the nose bridge region combined with reduced pressure at ear contact portions indicates forward head posture conditions, neck inclination conditions, or slouching conditions associated with the user.

20. The smart eyewear system as claimed in claim 15, wherein the controller is configured to establish the one or more threshold values during a calibration period using sensor data corresponding to normal user behavior or preferred ergonomic conditions collected over a predetermined duration.

21. The smart eyewear system as claimed in claim 15, wherein the controller is configured to dynamically adjust the one or more threshold values according to confidence scores associated with reliability characteristics of the sensor data, wherein sensor data having low confidence scores, inconsistent measurements, or transient anomalies are excluded or assigned reduced weighting during threshold adaptation operations.

22. A method for ergonomic cognitive strain management using a smart eyewear system, the method comprising:
collecting, by a plurality of sensors integrated into an eyewear frame, sensor data associated at least with posture conditions, neck positioning conditions, attention features, and interaction conditions associated with a user wearing the smart eyewear system;
processing, by a controller, the sensor data to generate synchronized posture features and attention features;
checking, by the controller, whether previously stored user specific baseline information corresponding to the user is available;
retrieving, by the controller, a previously stored user specific baseline associated with the user when the previously stored user specific baseline information is available, or determining, by the controller, a user specific baseline associated with the user when the previously stored user specific baseline information is unavailable;
determining, by the controller, an ergonomic cognitive strain state associated with the user based on the synchronized posture features, the attention features, and the user specific baseline; and
generating, by the controller, a feedback cue based on the ergonomic cognitive strain state and the user specific baseline, and outputting, by the controller, the feedback cue at the smart eyewear system.

23. The method as claimed in claim 22, further comprising:
generating, by one or more pressure sensors positioned at a nose bridge region and ear contact portions associated with an eyewear frame, pressure data;
generating, by at least one inertial sensing sensor, inertial data associated with head orientation and neck positioning conditions associated with the user;
generating, by at least one gaze or attention monitoring sensor, gaze related data and attention related data associated with the user;
temporally aligning and fusing, by the controller, the pressure data, the inertial data, the gaze related data, and the attention related data to determine an ergonomic cognitive strain state associated with the user; and
generating, by the controller, the feedback cue based on the ergonomic cognitive strain state and the user specific baseline.

24. The method as claimed in claim 23, further comprising:
generating, by the one or more pressure sensors, pressure distribution data and pressure differential data associated with the pressure data; and
determining, by the controller, posture imbalance conditions and neck positioning conditions using the pressure distribution data and the pressure differential data, wherein greater pressure at a left nose contact region relative to a right nose contact region or greater pressure at the right nose contact region relative to the left nose contact region indicates head tilt conditions or lateral imbalance conditions, and increased pressure at the nose bridge region combined with reduced pressure at ear contact portions indicates forward head posture conditions, neck inclination conditions, or slouching conditions associated with the user.

25. The method as claimed in claim 22, further comprising:
adapting, by the controller, the user specific baseline by dynamically adjusting one or more threshold values associated with posture characteristics, attention characteristics, or ergonomic cognitive strain characteristics based on historical sensor data and user interaction conditions associated with the user.

26. The method as claimed in claim 25, further comprising:
establishing, by the controller, the one or more threshold values during a calibration period using sensor data corresponding to normal user behavior or preferred ergonomic conditions collected over a predetermined duration.

27. The method as claimed in claim 25, further comprising:
adjusting, by the controller, the one or more threshold values according to confidence scores associated with reliability characteristics of the sensor data, wherein sensor data having low confidence scores, inconsistent measurements, or transient anomalies are excluded or assigned reduced weighting during threshold adaptation operations.

28. The method as claimed in claim 22, further comprising:
generating, by at least one electroencephalography (EEG) sensing sensor, cognitive activity related data associated with the user.

29. The method as claimed in claim 22, further comprising:
generating, by at least one ambient light sensor, environmental lighting data associated with a surrounding environment of the user.

30. A non-transitory computer-readable medium storing program instruction for ergonomic cognitive strain management, the program instructions, when executed, perform steps of:
collecting sensor data from a plurality of sensors integrated into an eyewear frame, the sensor data associated at least with posture conditions, neck positioning conditions, attention features, and interaction conditions associated with a user wearing smart eyewear system;
generating synchronized posture features and attention features by processing the sensor data;
checking availability of previously stored user specific baseline information corresponding to the user;
retrieving a previously stored user specific baseline associated with the user based on availability of the previously stored user specific baseline information, or determining a user specific baseline associated with the user based on unavailability of the previously stored user specific baseline information;
determining an ergonomic cognitive strain state associated with the user based on the synchronized posture features, the attention features, and the user specific baseline; and
generating a feedback cue based on the ergonomic cognitive strain state and the user specific baseline and outputting the feedback cue at the smart eyewear system.
